(19) United States
(12) Patent Application Publication
(10) Pub. No.: US 2023/0238114 A1
(43) Pub. Date: Jul. 27, 2023

(54) APPLIED BEHAVIORAL THERAPY APPARATUS AND METHOD

(71) Applicant: Yiftah Frechter, Irvington, NY (US)

(72) Inventor: Yiftah Frechter, Irvington, NY (US)

(21) Appl. No.: 17/648,926

(22) Filed: Jan. 25, 2022

(51) Int. Cl. G16H 20/70; G06V 20/40; G06V 40/16; G06F 3/0488; G10L 17/06; G10L 25/63; G16H 40/67; G16H 15/00

(52) U.S. Cl. CPC G16H 20/70; G06V 20/41; G06V 40/16; G06F 3/0488; G10L 17/06; G10L 25/63; G16H 40/67; G16H 15/00

(57) ABSTRACT

An apparatus for providing automated analysis and monitoring of an ABT session is presented herein. The apparatus may include a display configured to present material for the ABT session to a patient, at least one video capture device configured to capture video data for the ABT session related to at least one of first facial features of the patient, second facial features of a therapist, or a response to the material presented on the display, at least one audio capture device configured to capture audio data for the ABT session related to at least one of a first voice of the patient or a second voice of the therapist, and at least one processor configured to analyze, for the ABT session, data regarding the material presented on the display, the captured video data, and the captured audio data to produce an analysis of the ABT session.

### BACKGROUND

### Field

The present disclosure is generally directed to an apparatus for providing applied behavioral therapy (ABT).

### Related Art

In practice, quantifying the success of ABT sessions for some applications, e.g., the treatment of autism spectrum disorders, may be subjective. Additionally, the effectiveness of particular ABT interventions may be difficult to quantify. For example, in treating autism, ABT sessions may be conducted to train a patient to perform certain tasks or to respond appropriately to social cues. A therapist may subjectively measure the success of the patient on different tasks (e.g., ABT interventions) in the course of one or multiple sessions, but may not be able to produce objective data regarding the efficacy of the ABT interventions or the patient's progress over time.

### SUMMARY

Example implementations described herein include an innovative method for providing automated analysis and monitoring of an ABT session. The method may include presenting material for the ABT session to a patient via a display. The method may further include capturing video data for the ABT session related to at least one of first facial features of the patient, second facial features of a therapist, or a response to the material presented. The method may also include capturing audio data for the ABT session related to at least one of a first voice of the patient or a second voice of the therapist. The method may also include analyzing, for the ABT session, data regarding the material presented, the captured video data, and the captured audio data to produce an analysis of the ABT session.

Example implementations described herein include an innovative apparatus for providing automated analysis and monitoring of an ABT session. The apparatus may include a display configured to present material for the ABT session to a patient. The apparatus may further include at least one video capture device configured to capture video data for the ABT session related to at least one of first facial features of the patient, second facial features of a therapist, or a response to the material presented on the display. The apparatus may also include at least one audio capture device configured to capture audio data for the ABT session related to at least one of a first voice of the patient or a second voice of the therapist. The apparatus may further include at least one processor configured to analyze, for the ABT session, data regarding the material presented on the display, the captured video data, and the captured audio data to produce an analysis of the ABT session.

### BRIEF DESCRIPTION OF DRAWINGS

FIG. 1 shows an example of the architecture of an ABT provision and analysis system, in accordance with aspects of the invention.

FIG. 2A illustrates a first table-style embodiment of an ABT system that includes a display that is configured to present material for an ABT session to a patient.

FIG. 2B illustrates a second wall-style embodiment of an ABT system that includes a display that may include one or more monitors or a projected display that is configured to present material for an ABT session to a patient.

FIG. 3 illustrates a table-style embodiment of an ABT system that includes a display that is configured to present material for an ABT session to a patient.

FIG. 4 is a flowchart for a method of authenticating a user for an ABT session.

FIG. 5 is a flowchart for a method of providing automated analysis and monitoring of an ABT session.

FIG. 6 is a flowchart for a method of providing automated analysis and monitoring of an ABT session.

FIG. 7 illustrates an example computing environment with an example computer device suitable for use in some example implementations.

### DETAILED DESCRIPTION

The following detailed description provides details of the FIGs. and example implementations of the present application. Reference numerals and descriptions of redundant elements between FIGs. are omitted for clarity. Terms used throughout the description are provided as examples and are not intended to be limiting. For example, the use of the term “automatic” may involve fully automatic or semi-automatic implementations involving user or administrator control over certain aspects of the implementation, depending on the desired implementation of one of the ordinary skills in the art practicing implementations of the present application. Selection can be conducted by a user through a user interface or other input means, or can be implemented through a desired algorithm. Example implementations as described herein can be utilized either singularly or in combination and the functionality of the example implementations can be implemented through any means according to the desired implementations.

Example implementations described herein relate to an innovative method for providing automated analysis and monitoring of an ABT session. The method may include presenting material for the ABT session to a patient via a display. The method may further include capturing video data for the ABT session related to at least one of first facial features of the patient, second facial features of a therapist, or a response to the material presented. The method may also include capturing audio data for the ABT session related to at least one of a first voice of the patient or a second voice of the therapist. The method may also include analyzing, for the ABT session, data regarding the material presented, the captured video data, and the captured audio data to produce an analysis of the ABT session.

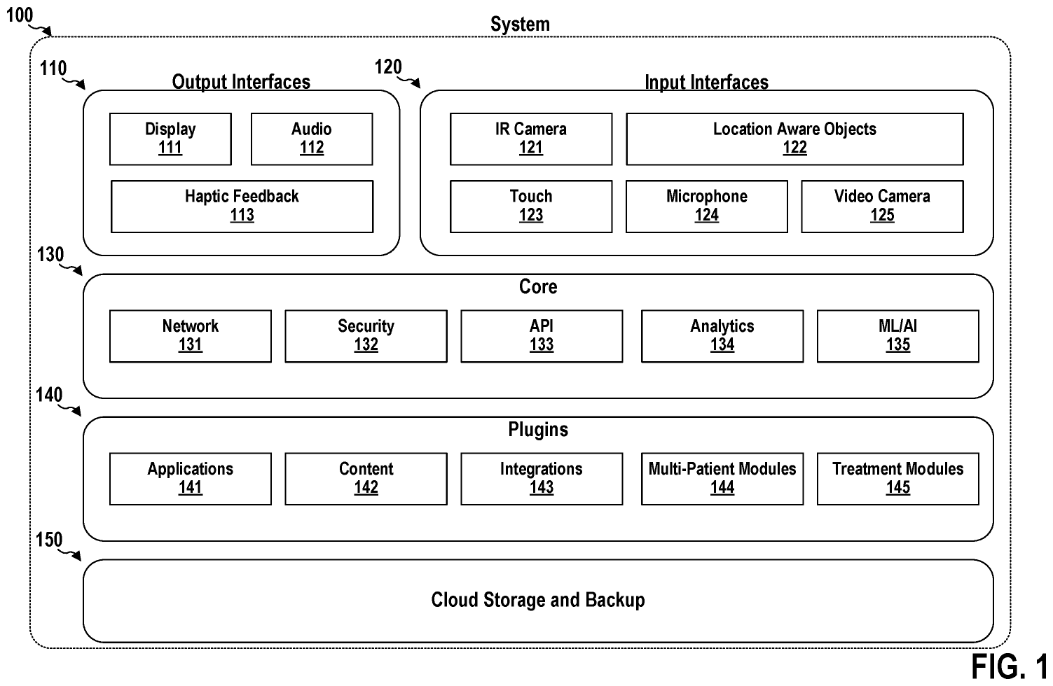

In some aspects, the innovative method may be performed as part of an ABT system to improve the provision and analysis of ABT, e.g., for autism. FIG. 1 shows an example of the architecture of an ABT provision and analysis system 100, in accordance with aspects of the invention. The ABT provision and analysis system 100, in some aspects, includes a set of output interfaces 110. The set of output interfaces, in some aspects, includes a display 111, an audio output 112, and a haptic feedback 113. The display 111 and audio output 112 may be configured to present material for an ABT session to a patient. The material presented for the ABT session may include haptic (e.g., vibrational) feedback or prompts via haptic feedback 113. The material presented during an ABT session may be related to tasks or drills for the treatment of autism. For example, the presented materials may introduce a user to social cues and appropriate responses or to common tasks that are often challenging for a user with an autism spectrum disorder.

The system, in some aspects, includes a set of input interfaces 120. The set of input interfaces 120 may include an infrared (IR) camera 121, a set of location aware objects 122, a touch sensitive component (e.g., a touch screen) 123, a microphone 124, and a video camera 125.

In some aspects, the location aware objects 122 may include objects configured to be placed on top of a display screen (e.g., display 111) to interact with the presented material for the ABT session. In order to establish a complete interactive learning and treatment ABT session for a patient, dedicated objects (e.g., location aware objects 122), in some aspects, may be placed on top of the screen. The screen and/or the location aware objects 122 may interact such that the ABT provision and analysis system 100 may determine an absolute position on the surface (e.g., a surface of the display 111) and relative position next to each other. These location aware objects 122, in some aspects, may represent real life objects related to specific tasks associated with the ABT session. For example, a location aware fork and knife may be provided in association with a task of the ABT session related to learning how to properly use cutlery. Other such location aware objects 122 may be provided in relation with other tasks associated with the ABT session.

A touch sensitive component 123, in some aspects, may include a capacitive touch flat surface that is capable of multi-touch and gestural input and/or interactions. In some aspects, the touch sensitive component 123 may be integrated into display 111. A screen size may vary and may take the form of a desktop or a wall. In some aspects, the screen (e.g., display 111) may be configured with sufficient resolution (e.g., HD,4K) to show videos, and smooth digital animation.

The microphone 124 may include one or more microphones that cover a room or an area in which an ABT session is performed. The microphone 124 may be configured to capture audio data for the ABT session related to at least one of a first voice of the patient or a second voice of the therapist. The captured audio may be processed by an analytics module 134 or an ML/AI module 135 using tonal analysis algorithms to, e.g., determine changes in a mood or a stress level of the patient and/or therapist or some other cognitive aspects.

Video camera 125 may include one or more cameras that cover the wide angles around the ABT provision and analysis system 100 as well as tracking the faces of both therapist and the patient. The video camera(s) 125 may be configured to capture video and/or still images of movements of both parties (e.g., the therapist and/or the patient) inside the room. The captured video and/or still images may be processed by an analytics module 134 or an ML/AI module 135 using facial analysis algorithms to, e.g., determine changes in their mood or some other cognitive aspects.

The ABT provision and analysis system 100 may include a set of core modules 130. The set of core modules may include a network module 131 that interacts with a local or external network. For example, the network module 131 may be configured to communicate with an external system storing medical records in a secure environment (e.g., protected according to Health Insurance Portability and Accountability Act (HIPAA) standards). The set of core modules 130 may further include a security module 132 that may authenticate a user (e.g., a patient and/or therapist) for an ABT session. For example, before an ABT session starts, a therapist may be required to login to the system. The login may allow the system to register information collected for the ABT session to the right patient and therapist as well as to retrieve historic data about the patient and/or therapist that serves as a baseline for current session.

Station units that are connected to an EMR (electronic medical records) system (e.g., via network module 131) or clinic scheduling system will be able to initialize themselves based on predefined schedule but will still require authentication login as schedule and rooms may change and a patient's data is under HIPAA requirement of privacy. Since all gathered information, in some aspects, may be under HIPAA strict privacy protocols, authenticating is a prerequisite to any treatment using the ABT provision and analysis system 100. The security module 132 may use sensors of the ABT provision and analysis system 100 for any or all of a set of authentication methods. In some aspects, the authentication methods may include facial recognition, voice authentication, fingerprint authentication, or password authentication.

For example, facial recognition may be performed by software modules (e.g., security module 132) of an operating system (e.g., an OS executing the set of core modules 130) that may be configured to detect authorized faces. The ABT provision and analysis system 100, in some aspects, may use its dedicated video camera(s) 125 and/or IR cameras 121 and publicly available algorithms for face recognition and analyses to allow for seamless and secured authentication for the therapist.

In some aspects, the ABT provision and analysis system 100 may be deployed and/or operated in a controlled environment such as a clinic or home. Accordingly, voice biometric authentication may be an option even though it may not be secure enough in public spaces. The ABT provision and analysis system 100, in some aspects, may use the microphone 124 and analyze the biometric patterns of the voice to match against authorized users that registered for voice based authentication. Some ABT provision and analysis system 100 models will include a fingerprint sensor that can be used to authenticate and authorize approved therapist login. Fingerprint sensors and their software are very reliable and may be publicly and commercially available for integration. In some aspects, the ABT provision and analysis system 100 may support username password authentication. Passwords, in some aspects, may be input using the system touch keyboard (e.g., touch sensitive component 123) and will be matched against an encrypted authentication database. In some aspects, by default options like “remember password” are turned off given that most systems are shared and not private.

The set of core modules 130 may include an application programming interface (API) or a set of APIs 133. The APIs may expose functions for interactions between the set of output interfaces 110, the set of input interfaces 120, the set of core modules 130, a set of plugins 140, and a cloud storage and backup 150. The set of core modules may include an analytics module 134 that may be responsible for fetching past sessions data as well as real time data from the current session and preparing reports, e.g., a visual presentation in the form of a dashboard, charts, and/or tables. The reports, in some aspects, may be used to share information with other professionals in the clinic such as other therapists as well as outside the clinic such as referring physicians. A simplified, easier-to-understand version of the analytics report may be provided to a patient's caregiver in order to track the patient's progress. In some aspects, the set of APIs 133 may be responsible for exposing the ABT provision and analysis system 100 core capabilities to 3rd party applications, plugins, and integrations developers. For example, new drills may be added by 3rd party developers using the set of APIs 133. The set of APIs 133, in some aspects, may allow data to be pushed into, or fetched from, the ABT provision and analysis system 100 using dedicated API calls.

The set of core modules 130 may further include a ML/AI module 135 for processing captured video or audio data. The ML/AI module 135, in some aspects, may analyze captured video data to identify patient and/or therapist responses, attentional state (e.g., whether, and to what, a patient and/or therapist is paying attention), or an emotional state during an ABT session. In some aspects, the ML/AI module 135 may also analyze captured audio data to perform a tonal analysis to identify a stress level of the patient and/or the therapist. In some aspects, the ML/AI module 135 may be responsible for receiving real time data from sensors (e.g., any of the inout interfaces in the set of input interfaces 120) and correlating it with past data for immediate actions within the session. An example would be to correlate real time data from a user's face and voice (e.g., based on facial and voice recognition) to determine if the patient is having difficulty keeping up with the drills or tasks and adjusting the session difficulty during the session. ML/AI module 135, in some aspects, may compare current patient data with historical and baseline patient data for every drill.

The ABT provision and analysis system 100 may also include a set of plugins 140. The set of plugins 140 may include a set of applications 141, a set of content 142, a set of integrations 143, a set of multi-patient modules 144, and a set of treatment modules 145. The set of plugins 140 may include 3^{rd} party applications, content modules, integrations, or plugins developed for the ABT provision and analysis system 100 to use the set of APIs 133.

ABT provision and analysis system 100 may be implemented as a “behave station” for providing ABT for the treatment of autism in both clinical and hone environments. Accordingly, the ABT provision and analysis system 100 (or behave station) may be purchased or leased for both clinic usage in which it is shared among several clinic patients or alternatively for home use in which it is used for a single patient or possibly siblings. Home use, in some aspects, may allow for a more “relaxed” security and authentication requirements.

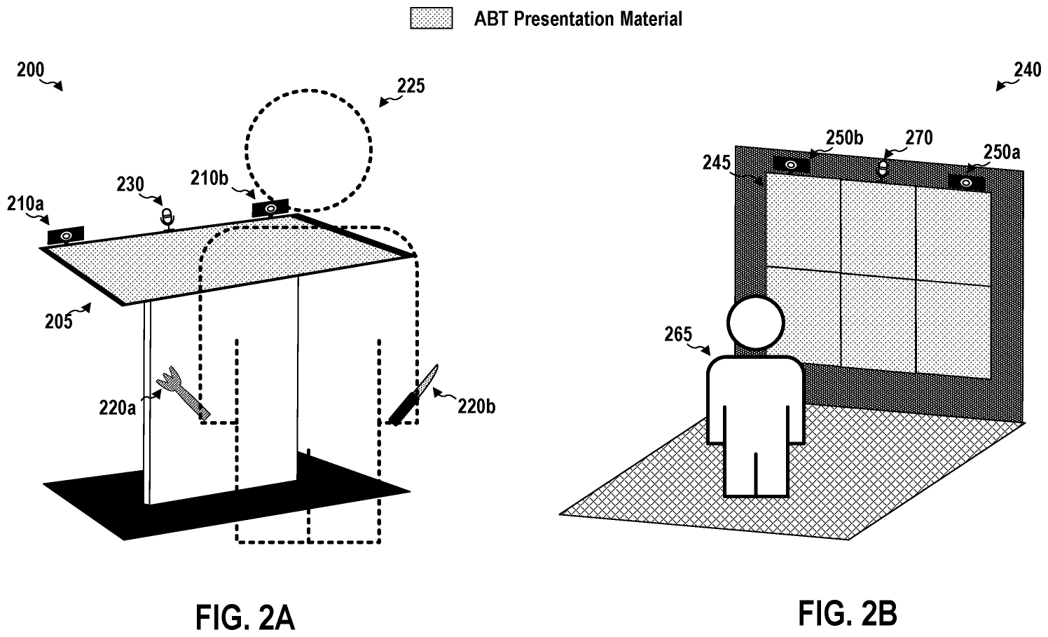

FIGS. 2A and 2B illustrates a first and second embodiments of an ABT apparatus 200 and 240 that illustrate two embodiments of an ABT system (e.g., implementations of an ABT provision and analysis system 100). FIG. 2A illustrates a first table-style embodiment of an ABT apparatus 200 that includes a display 205 that is configured to present material for an ABT session to a user (e.g., a patient and/or a therapist) 225. As described in relation to FIG. 1, the ABT apparatu 200 may include a set of video cameras 210a and 210b, a set of location aware objects 220a and 220b, and a microphone 230.

Similarly, FIG. 2B illustrates a second wall-style embodiment of an ABT apparatus 240 that includes a display 245 that may include one or more monitors or a projected display that is configured to present material for an ABT session to a user (e.g., a patient and/or a therapist) 265. The second wall-style embodiment of the ABT apparatus 240 may also include a set of cameras 250a and 250b and a microphone 270.

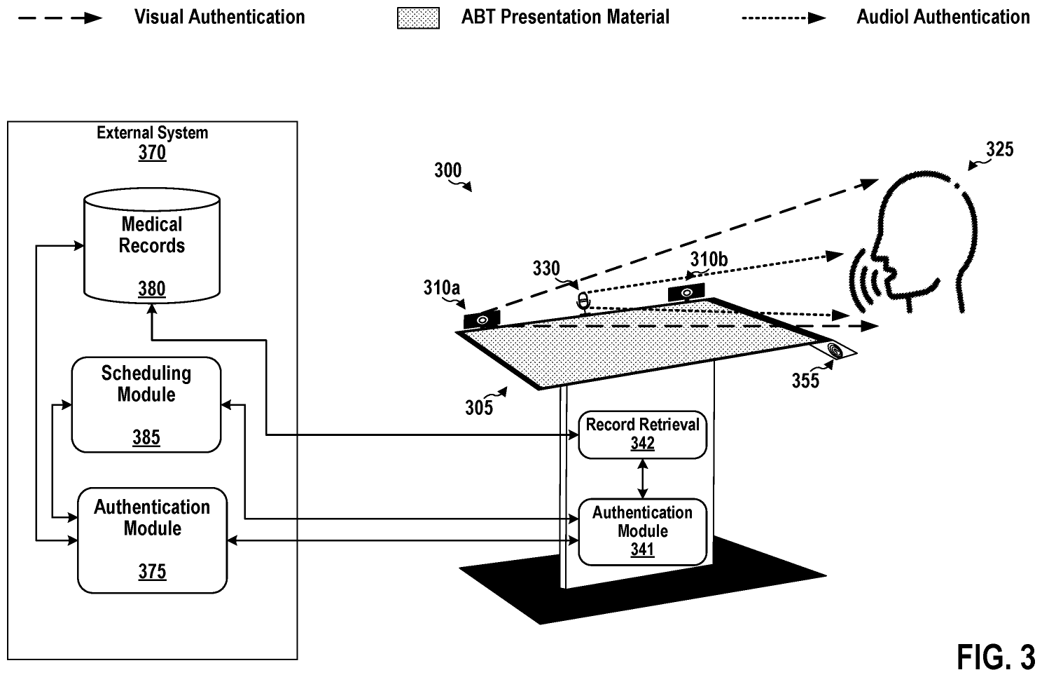

FIG. 3 illustrates a table-style embodiment of an ABT apparatus 300 that includes a display 305 that is configured to present material for an ABT session to a user (e.g., a patient and/or a therapist) 325. As described in relation to FIG. 1, the table-style embodiment of an ABT apparatus 300 may include a set of video cameras 310a and 310b, a microphone 230, and a fingerprint module 355. The table-style embodiment of an ABT apparatus 300 may include an authentication module 341 (e.g., implemented by security module 132 and/or ML/AI 135) that interacts with one or more of the set of video cameras 310a and 310b, a microphone 230, and a fingerprint module 355, to authenticate a user (e.g., a patient and/or a therapist) 325. The authentication process may be initiated by a user attempting to login to an ABT session (e.g., requesting an ABT session).

In some aspects, an authentication process may be initiated based on a scheduling module 385 of the external system 370 that is aware of a planned schedule of ABT sessions. The table-style embodiment of an ABT apparatus 300 may interact with an external system 370. The external system 370 may include an authentication module 375 that interacts with the authentication module 341 of the table-style embodiment of an ABT apparatus 300 to authenticate the user and/or the table-style embodiment of an ABT apparatus 300 to access a set of medical records 380 associated with a requested ABT session.

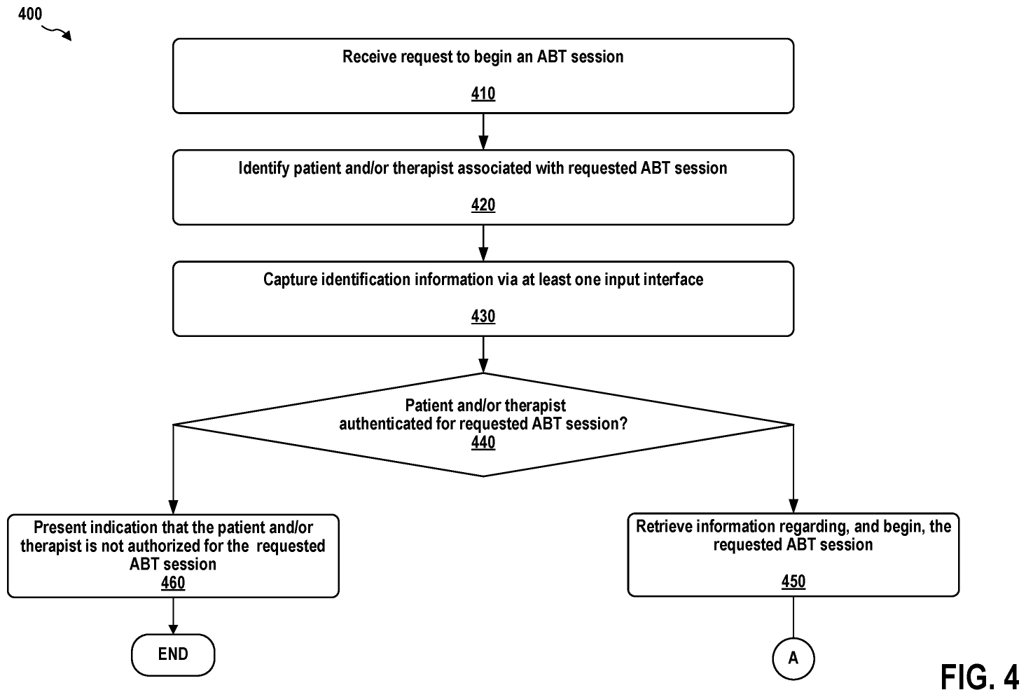

FIG. 4 is a flowchart 400 for a method of authenticating a user for an ABT session. The method may be performed by an ABT apparatus (e.g., ABT provision and analysis system 100; ABT apparatus 200, 240, and 300). At 410 the ABT apparatus, and more specifically an authentication module of the ABT apparatus, may receive a request to begin an ABT session. The request may be received from a user or from an external scheduling module. A request received from a user may include an attempted login attempt. For example, referring to FIG. 3, an ABT apparatus 300 may receive, at an authentication module 341, a request from one of a user (e.g., a patient and/or a therapist) 325 or from a scheduling module 385 of an external system 370.

At 420, the ABT apparatus, and more specifically an authentication module of the ABT apparatus, may identify one or more of a patient or a therapist associated with the requested ABT session. The identification may be based on an input from the user or from the external scheduling module. For example, a therapist may enter a session identifier, a therapist identifier, a patient identifier, or a therapist may login as a therapist and then select a patient and/or an ABT session. For example, referring to FIGS. 1 and 3, the ABT provision and analysis system 100 or ABT apparatus 300 may receive input via one or more of the interfaces 120 (e.g., touch sensitive component 123, microphone 124, etc.) to identify a user (e.g., a therapist or patient) 325.

At 430, the ABT apparatus, and more specifically an authentication module of the ABT apparatus, may capture identification information via at least one input interface. For example, the ABT apparatus may capture one or more of a password, a voice for audio recognition, an image for facial recognition, or a fingerprint. For example, referring to FIGS. 1 and 3, the ABT provision and analysis system 100 or ABT apparatus 300 may capture a password via a touch sensitive component 123, audio via a microphone 124 or microphone 330, a video camera 125 or camera 310a (or 310b), or a fingerprint module 355.

At 440, the ABT apparatus, and more specifically an authentication module of the ABT apparatus, may determine whether a patient and/or therapist (identified at 420) is authenticated for the requested ABT session. The authentication may include comparing the identification information captured at 430 to one or more of a voiceprint, a faceprint (e.g., facial recognition data), a fingerprint, or a password associated with the identified patient and/or therapist. In some aspects, the authentication may include communicating with an external authentication server. For example, referring to FIG. 3, an authentication module 341 of an ABT apparatus 300 may determine whether identification information captured by one of a camera 310a (or 310b), a microphone 330, a fingerprint module 355, or a touch sensitive display 305 matches identification information stored for a patient and/or a therapist associated with a requested ABT session. In some aspects, the authentication module 341 of an ABT apparatus 300 may determine whether identification information captured by one of a camera 310a (or 310b), a microphone 330, a fingerprint module 355, or a touch sensitive display 305 matches identification information stored for a patient and/or a therapist associated with a requested ABT session by transmitting the captured identification to an authentication module 375.

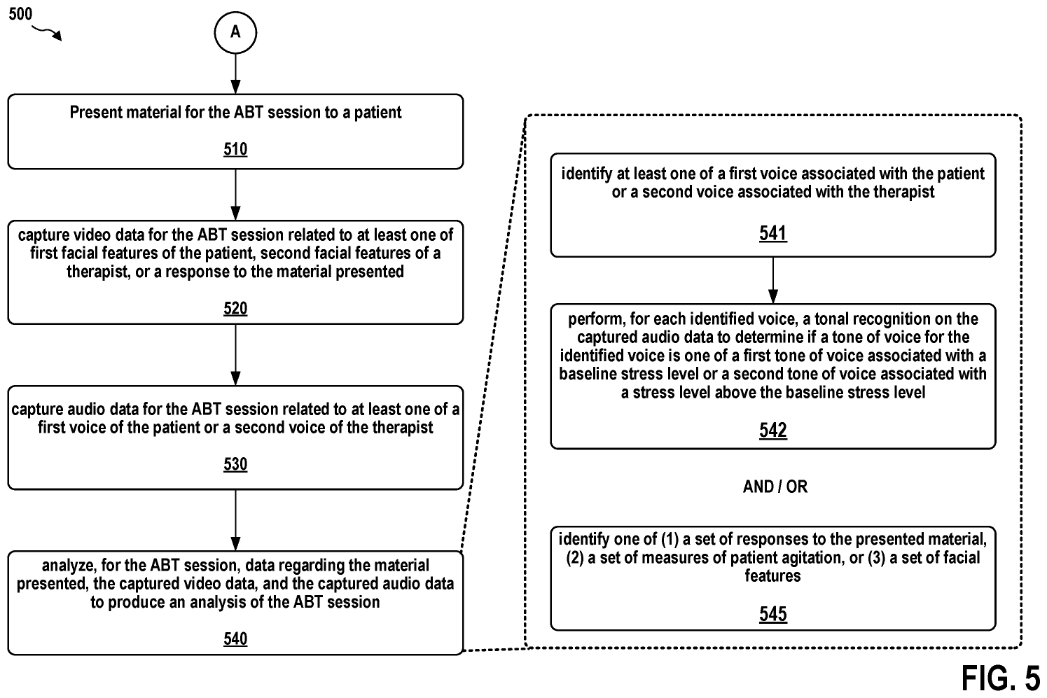

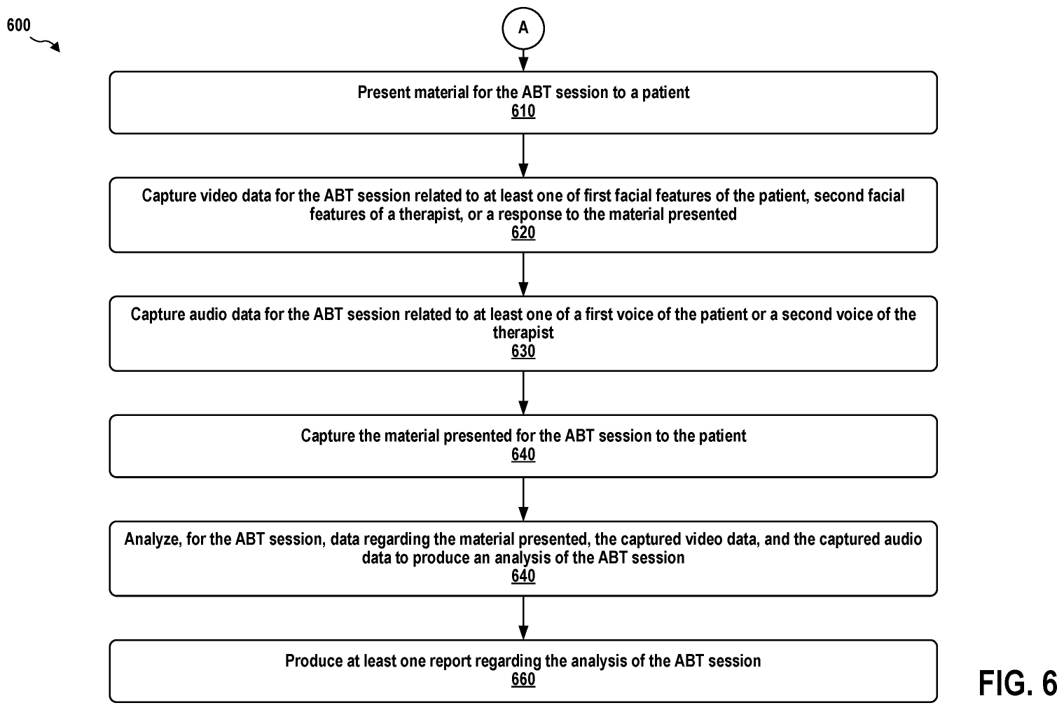

If the ABT apparatus determines, at 440, that the patient and/or therapist is authenticated for the requested ABT session, the ABT session may, at 450, retrieve information regarding, and begin, the requested ABT session. The ABT apparatus may proceed to one of FIG. 5 or 6 as indicated by the circled “A” in FIGS. 4, 5, and 6. For example, referring to FIG. 3, the ABT apparatus 300 may retrieve records via record retrieval module 342. The record retrieval module 342 may retrieve information regarding the ABT session from a local storage or from an external storage such as stored medical records 380 in external system 370.

If the ABT apparatus determines, at 440, that the patient and/or therapist is not authenticated for the requested ABT session, the ABT apparatus may present, at 460, an indication that the patient and/or therapist is not authorized for the requested ABT session. Presenting, at 460, the indication may include presenting an option to request an additional ABT session or to input additional identification information, and the process ends.

FIG. 5 is a flowchart 500 for a method of providing automated analysis and monitoring of an ABT session. The method may be performed by an ABT apparatus (e.g., ABT provision and analysis system 100; ABT apparatus 200, 240, and 300). At 510, the ABT apparatus may present material for the ABT session to a patient via a display. In some aspects, the display may be a touch-sensitive display. The display, in some aspects, may be one or more of a tabletop display, a monitor, a set of monitors, a wall-mounted display, or a projected display. The display may interact with a set of interactive accessories (e.g., location-aware accessories). As discussed above in relation to FIG. 1, the material presented may be related to the treatment of an autism spectrum disorder and may include specific drills and/or tasks that provide a behavioral intervention for a patient on the autism spectrum. For example, referring to FIGS. 2A, 2B, and 3, ABT apparatus 200, 240, or 300 may present material for the ABT session via display 205, 245, or 305 and may interact with a set of interactive accessories (e.g., location aware objects 220a and/or 220b).

At 520, the ABT apparatus may capture video data for the ABT session related to at least one of first facial features of the patient, second facial features of a therapist, or a response to the material presented. In some aspects, the captured video data may include a set of patient and/or therapist feedback and/or responses to the material presented at 510 for adjusting the presented material or progressing through the ABT session. For example, referring to FIGS. 2A, 2B, and 3, ABT apparatus 200, 240, or 300 may capture video data using one or more of cameras 210a or 210b, cameras 250a or 250b, or cameras 310a or 310b, respectively. The captured video data may include data relating to a user (e.g., a patient or a therapist) 225, 265, or 325.

At 530, the ABT apparatus may capture audio data for the ABT session related to at least one of a first voice of the patient or a second voice of the therapist. The captured audio data may include spoken responses to presented materials, an instruction from a therapist to a patient, a response from the patient to the therapist, or some other interaction between a patient and a therapist. For example, referring to FIGS. 2A, 2B, and 3, ABT apparatus 200, 240, or 300 may capture audio data using microphone 230, 270, or 330, respectively from a user (e.g., a patient and/or a therapist) 225, 265, or 325.

At 540, the ABT apparatus may analyze, for the ABT session, data regarding the material presented, the captured video data, and the captured audio data to produce an analysis of the ABT session. The analysis may include one or more of single-mode analysis and multi-mode analysis. The analysis, in some aspects, may be performed by multiple programs and/or methodologies. For example, a first analytics program of the ABT apparatus may be used to perform a speech-to-text operation, while a tonal analysis may be performed by a machine-trained program (e.g., commonly referred to as machine learning, neural networks, artificial intelligence, etc.). A machine-trained program may be trained for each patient and/or therapist and a set of trained parameters (e.g., weights of neurons in a neural network) may be used based on the ABT session being associated with a particular patient and/or therapist. The ABT apparatus may perform the analysis, in some aspects, by transmitting the captured data to an analysis server or cloud-based analysis tool.

For example, a single-mode analysis may be performed on the audio data captured at 530 to identify, at 541, at least one of a first voice associated with the patient or a second voice associated with the therapist and, at 542 perform, for each identified voice, a tonal recognition on the captured audio data to determine if a tone of voice for the identified voice is one of a first tone of voice associated with a baseline stress level or a second tone of voice associated with a stress level above the baseline stress level. In some aspects, the single-mode analysis may include a speech to text recognition used to generate a transcript of the ABT session.

Similarly, a single-mode analysis for video data captured at 520 may include, at 545, identifying one of (1) a set of responses to the presented material, (2) a set of measures of patient agitation, or (3) a set of facial features. The set of responses to the presented material, in some aspects, may include one or more of following an instruction presented on the display or selecting one of multiple options presented on the display. The set of facial features may be used, for example, to determine an emotional state, to determine an attentional state (e.g., whether, or to what, the patient is paying attention), or to perform a facial recognition, among other functions.

A multi-mode analysis may process two or more temporally-correlated data sets (e.g., data associated with a same time-stamp) captured at 520 and/or 530 or data regarding the presented material temporally-associated with the captured data. The multi-mode analysis may be based on the output of single-mode analysis for each temporally-correlated data sets being processed or may be based on the data itself. For example, a multi-mode analysis may process data regarding material presented via the display, an emotional or attentional state identified by a single mode analysis, and an identified response to a task associated with the material presented via the display to identify a measure of success associated with the task or an absolute, or relative, efficacy of a particular approach. An efficacy may be determined, e.g., by comparing a current measure of success against past measures of success for similar tasks and/or calculating a rate of improvement and comparing it to a rate of improvement associated with other approaches. For example, referring to FIG. 1, the ABT provision and analysis system 100, and specifically analytics module 134 and ML/AI module 135, may perform an analysis of data captured based on one or more of IR camera 121, location aware objects 122, touch sensitive component 123, microphone 124, or video camera 125.

In some aspects, the analysis of the ABT session may include at least one of a first analysis of therapist efficacy, a second analysis of the efficacy of different material presented on the display, or third analysis of a progress of the patient. As discussed above the analysis may be based on more than one of the data regarding the material presented on the display, the captured video data, and the captured audio data. The analysis may then be used to produce (or generate) at least one report regarding the analysis of the ABT session. For example, the ABT apparatus may generate a set of reports for a set of users, e.g., a therapist, a supervising therapist, a parent, an insurer, and so on. The reports may include data that is identified as being useful to the user receiving the report and not violating the HIPAA.

FIG. 6 is a flowchart 600 for a method of providing automated analysis and monitoring of an ABT session. The method may be performed by an ABT apparatus (e.g., ABT provision and analysis system 100; ABT apparatus 200, 240, and 300). At 610, the ABT apparatus may present material for the ABT session to a patient via a display. In some aspects, the display may be a touch-sensitive display. The display, in some aspects, may be one or more of a tabletop display, a monitor, a set of monitors, a wall-mounted display, or a projected display. The display may interact with a set of interactive accessories (e.g., location-aware accessories). For example, referring to FIGS. 2A, 2B, and 3, ABT apparatus 200, 240, or 300 may present material for the ABT session via display 205, 245, or 305 and may interact with a set of location aware objects 220a and/or 220b.

At 620, the ABT apparatus may capture video data for the ABT session related to at least one of first facial features of the patient, second facial features of a therapist, or a response to the material presented. In some aspects, the captured video data may include a set of patient and/or therapist feedback and/or responses to the material presented at 610 for adjusting the presented material or progressing through the ABT session. For example, referring to FIGS. 2A, 2B, and 3, ABT apparatus 200, 240, or 300 may capture video data using one or more of cameras 210a or 210b, cameras 250a or 250b, or cameras 310a or 310b, respectively. The captured video data may include data relating to a user (e.g., a patient or a therapist) 225, 265, or 325.

At 630, the ABT apparatus may capture audio data for the ABT session related to at least one of a first voice of the patient or a second voice of the therapist. The captured audio data may include spoken responses to presented materials, an instruction from a therapist to a patient, a response from the patient to the therapist, or some other interaction between a patient and a therapist. For example, referring to FIGS. 2A, 2B, and 3, ABT apparatus 200, 240, or 300 may capture audio data using microphone 230, 270, or 330, respectively from a user (e.g., a patient and/or a therapist) 225, 265, or 325.

At 640, the ABT apparatus may capture the material presented for the ABT session to the patient. Capturing the material presented for the ABT session may include capturing interactions (e.g., selections) with the presented material. The captured material may be organized based on tasks, time stamps, or may be broken up into distinct phases (e.g., prompt, response, feedback, etc.). For example, referring to FIGS. 2A, 2B, and 3, ABT apparatus 200, 240, or 300 may capture material presented on display 205, 245, or 305, respectively.

At 650, the ABT apparatus may analyze, for the ABT session, data regarding the material presented, the captured video data, and the captured audio data to produce an analysis of the ABT session. The analysis may include one or more of single-mode analysis and multi-mode analysis. The analysis, in some aspects, may be performed by multiple programs and/or methodologies. For example, a first analytics program of the ABT apparatus may be used to perform a speech-to-text operation, while a tonal analysis may be performed by a machine-trained program (e.g., commonly referred to as machine learning, neural networks, artificial intelligence, etc.). A machine-trained program may be trained for each patient and/or therapist and a set of trained parameters (e.g., weights of neurons in a neural network) may be used based on the ABT session being associated with a particular patient and/or therapist. The ABT apparatus may perform the analysis, in some aspects, by transmitting the captured data to an analysis server or cloud-based analysis tool.

For example, a single-mode analysis may be performed on the audio data captured at 630 to identify at least one of a first voice associated with the patient or a second voice associated with the therapist and perform, for each identified voice, a tonal recognition on the captured audio data to determine if a tone of voice for the identified voice is one of a first tone of voice associated with a baseline stress level or a second tone of voice associated with a stress level above the baseline stress level. In some aspects, the single-mode analysis may include a speech to text recognition used to generate a transcript of the ABT session.

Similarly, a single-mode analysis for video data captured at 620 may include identifying one of (1) a set of responses to the presented material, (2) a set of measures of patient agitation, or (3) a set of facial features. The set of responses to the presented material, in some aspects, may include one or more of following an instruction presented on the display or selecting one of multiple options presented on the display. The set of facial features may be used, for example, to determine an emotional state, to determine an attentional state (e.g., whether, or to what, the patient is paying attention), or to perform a facial recognition, among other functions.

A multi-mode analysis may process two or more temporally-correlated data sets (e.g., data associated with a same time-stamp) captured at 620 and/or 630 or data regarding the presented material temporally-associated with the captured data. The multi-mode analysis may be based on the output of single-mode analysis for each temporally-correlated data sets being processed or may be based on the data itself. For example, a multi-mode analysis may process data regarding material presented via the display, an emotional or attentional state identified by a single mode analysis, and an identified response to a task associated with the material presented via the display to identify a measure of success associated with the task or an absolute, or relative, efficacy of a particular approach. An efficacy may be determined, e.g., by comparing a current measure of success against past measures of success for similar tasks and/or calculating a rate of improvement and comparing it to a rate of improvement associated with other approaches. For example, referring to FIG. 1, the ABT provision and analysis system 100, and specifically analytics module 134 and ML/AI module 135, may perform an analysis of data captured based on one or more of IR camera 121, location aware objects 122, touch sensitive component 123, microphone 124, or video camera 125.

In some aspects, the analysis of the ABT session may include at least one of a first analysis of therapist efficacy, a second analysis of the efficacy of different material presented on the display, or third analysis of a progress of the patient. As discussed above the analysis may be based on more than one of the data regarding the material presented on the display, the captured video data, and the captured audio data. The analysis may then be used, at 660, to produce (or generate) at least one report regarding the analysis of the ABT session. For example, the ABT apparatus may generate a set of reports for a set of users, e.g., a therapist, a supervising therapist, a parent, an insurer, and so on. The reports may include data that is identified as being useful to the user receiving the report and not violating the HIPAA.

By applying the invention disclosed above, the ABT system may provide enhanced analysis of ABT sessions. The ABT system may present material for an ABT session, record audio and visual data associated with the presented material. The ABT system may perform an analysis of the captured data to provide enhanced reporting as described above.

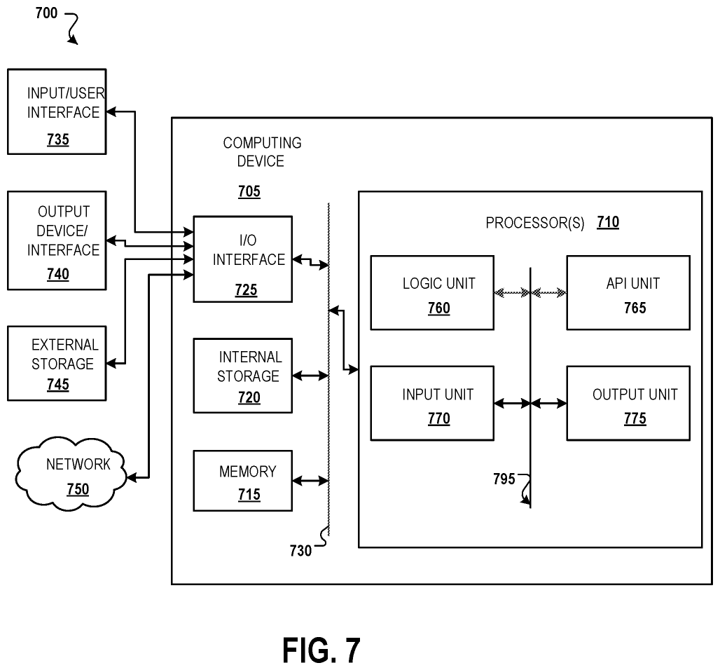

FIG. 7 illustrates an example computing environment with an example computer device suitable for use in some example implementations. Computer device 705 in computing environment 700 can include one or more processing units, cores, or processors 710, memory 715 (e.g., RAM, ROM, and/or the like), internal storage 720 (e.g., magnetic, optical, solid-state storage, and/or organic), and/or IO interface 725, any of which can be coupled on a communication mechanism or bus 730 for communicating information or embedded in the computer device 705. IO interface 725 is also configured to receive images from cameras or provide images to projectors or displays, depending on the desired implementation.

Computer device 705 can be communicatively coupled to input/user interface 735 and output device/interface 740. Either one or both of the input/user interface 735 and output device/interface 740 can be a wired or wireless interface and can be detachable. Input/user interface 735 may include any device, component, sensor, or interface, physical or virtual, that can be used to provide input (e.g., buttons, touch-screen interface, keyboard, a pointing/cursor control, microphone, camera, braille, motion sensor, accelerometer, optical reader, and/or the like). Output device/interface 740 may include a display, television, monitor, printer, speaker, braille, or the like. In some example implementations, input/user interface 735 and output device/interface 740 can be embedded with or physically coupled to the computer device 705. In other example implementations, other computer devices may function as or provide the functions of input/user interface 735 and output device/interface 740 for a computer device 705.

Examples of computer device 705 may include, but are not limited to, highly mobile devices (e.g., smartphones, devices in vehicles and other machines, devices carried by humans and animals, and the like), mobile devices (e.g., tablets, notebooks, laptops, personal computers, portable televisions, radios, and the like), and devices not designed for mobility (e.g., desktop computers, other computers, information kiosks, televisions with one or more processors embedded therein and/or coupled thereto, radios, and the like).

Computer device 705 can be communicatively coupled (e.g., via IO interface 725) to external storage 745 and network 750 for communicating with any number of networked components, devices, and systems, including one or more computer devices of the same or different configuration. Computer device 705 or any connected computer device can be functioning as, providing services of, or referred to as a server, client, thin server, general machine, special-purpose machine, or another label.

IO interface 725 can include but is not limited to, wired and/or wireless interfaces using any communication or IO protocols or standards (e.g., Ethernet, 702.11x, Universal System Bus, WiMax, modem, a cellular network protocol, and the like) for communicating information to and/or from at least all the connected components, devices, and network in computing environment 700. Network 750 can be any network or combination of networks (e.g., the Internet, local area network, wide area network, a telephonic network, a cellular network, satellite network, and the like).

Computer device 705 can use and/or communicate using computer-usable or computer readable media, including transitory media and non-transitory media. Transitory media include transmission media (e.g., metal cables, fiber optics), signals, carrier waves, and the like. Non-transitory media include magnetic media (e.g., disks and tapes), optical media (e.g., CD ROM, digital video disks, Blu-ray disks), solid-state media (e.g., RAM, ROM, flash memory, solid-state storage), and other non-volatile storage or memory.

Computer device 705 can be used to implement techniques, methods, applications, processes, or computer-executable instructions in some example computing environments. Computer-executable instructions can be retrieved from transitory media, and stored on and retrieved from non-transitory media. The executable instructions can originate from one or more of any programming, scripting, and machine languages (e.g., C, C++, C#, Java, Visual Basic, Python, Perl, JavaScript, and others).

Processor(s) 710 can execute under any operating system (OS) (not shown), in a native or virtual environment. One or more applications can be deployed that include logic unit 760, application programming interface (API) unit 765, input unit 770, output unit 775, and inter-unit communication mechanism 795 for the different units to communicate with each other, with the OS, and with other applications (not shown). The described units and elements can be varied in design, function, configuration, or implementation and are not limited to the descriptions provided. Processor(s) 710 can be in the form of hardware processors such as central processing units (CPUs) or in a combination of hardware and software units.

In some example implementations, when information or an execution instruction is received by API unit 765, it may be communicated to one or more other units (e.g., logic unit 760, input unit 770, output unit 775). In some instances, logic unit 760 may be configured to control the information flow among the units and direct the services provided by API unit 765, the input unit 770, the output unit 775, in some example implementations described above. For example, the flow of one or more processes or implementations may be controlled by logic unit 760 alone or in conjunction with API unit 765. The input unit 770 may be configured to obtain input for the calculations described in the example implementations, and the output unit 775 may be configured to provide an output based on the calculations described in example implementations.

Processor(s) 710 can be configured to present material for the ABT session to a patient via a display. The processor(s) 710 may also be configured to capture video data for the ABT session related to at least one of first facial features of the patient, second facial features of a therapist, or a response to the material presented. The processor(s) 710 may further be configured to capture audio data for the ABT session related to at least one of a first voice of the patient or a second voice of the therapist. The processor(s) 710 may further be configured to analyze, for the ABT session, data regarding the material presented, the captured video data, and the captured audio data to produce an analysis of the ABT session. The processor(s) 710 may also be configured to identify, when at least one interactive accessory is placed on a surface of the display, a position of the at least one interactive accessory relative to the presented material. The processor(s) 710 may also be configured to identify at least one of the first voice associated with the patient or the second voice associated with the therapist. The processor(s) 710 may also be configured to perform, for each identified voice, a tonal recognition on the captured audio data to determine if a tone of voice for the identified voice is one of a first tone of voice associated with a baseline stress level or a second tone of voice associated with a stress level above the baseline stress level. The processor(s) 710 may further be configured to capture the material presented for the ABT session to the patient. The processor(s) 710 may further be configured to identify one of (1) a set of responses to the presented material, (2) a set of measures of patient agitation, or (3) a set of facial features. The processor(s) 710 may further be configured to produce at least one report regarding the analysis of the ABT session. The processor(s) 710 may further be configured to process at least one of the data regarding the material presented on the display, the captured video data, and the captured audio data using one of a machine-trained network or a neural network.

Some portions of the detailed description are presented in terms of algorithms and symbolic representations of operations within a computer. These algorithmic descriptions and symbolic representations are the means used by those skilled in the data processing arts to convey the essence of their innovations to others skilled in the art. An algorithm is a series of defined steps leading to a desired end state or result. In example implementations, the steps carried out require physical manipulations of tangible quantities for achieving a tangible result.

Unless specifically stated otherwise, as apparent from the discussion, it is appreciated that throughout the description, discussions utilizing terms such as “processing,” “computing,” “calculating,” “determining,” “displaying,” or the like, can include the actions and processes of a computer system or other information processing device that manipulates and transforms data represented as physical (electronic) quantities within the computer system's registers and memories into other data similarly represented as physical quantities within the computer system's memories or registers or other information storage, transmission or display devices.

Example implementations may also relate to an apparatus for performing the operations herein. This apparatus may be specially constructed for the required purposes, or it may include one or more general-purpose computers selectively activated or reconfigured by one or more computer programs. Such computer programs may be stored in a computer readable medium, such as a computer readable storage medium or a computer readable signal medium. A computer readable storage medium may involve tangible mediums such as, but not limited to optical disks, magnetic disks, read-only memories, random access memories, solid-state devices, and drives, or any other types of tangible or non-transitory media suitable for storing electronic information. A computer readable signal medium may include mediums such as carrier waves. The algorithms and displays presented herein are not inherently related to any particular computer or other apparatus. Computer programs can involve pure software implementations that involve instructions that perform the operations of the desired implementation.

Various general-purpose systems may be used with programs and modules in accordance with the examples herein, or it may prove convenient to construct a more specialized apparatus to perform desired method steps. In addition, the example implementations are not described with reference to any particular programming language. It will be appreciated that a variety of programming languages may be used to implement the teachings of the example implementations as described herein. The instructions of the programming language(s) may be executed by one or more processing devices, e.g., central processing units (CPUs), processors, or controllers.

As is known in the art, the operations described above can be performed by hardware, software, or some combination of software and hardware. Various aspects of the example implementations may be implemented using circuits and logic devices (hardware), while other aspects may be implemented using instructions stored on a machine-readable medium (software), which if executed by a processor, would cause the processor to perform a method to carry out implementations of the present application. Further, some example implementations of the present application may be performed solely in hardware, whereas other example implementations may be performed solely in software. Moreover, the various functions described can be performed in a single unit, or can be spread across a number of components in any number of ways. When performed by software, the methods may be executed by a processor, such as a general-purpose computer, based on instructions stored on a computer readable medium. If desired, the instructions can be stored on the medium in a compressed and/or encrypted format.

Moreover, other implementations of the present application will be apparent to those skilled in the art from consideration of the specification and practice of the teachings of the present application. Various aspects and/or components of the described example implementations may be used singly or in any combination. It is intended that the specification and example implementations be considered as examples only, with the true scope and spirit of the present application being indicated by the following claims.

What is claimed:

1. An apparatus for providing automated analysis and monitoring of an applied behavioral therapy (ABT) session, the apparatus comprising:
a display configured to present material for the ABT session to a patient;
at least one video capture device configured to capture video data for the ABT session related to at least one of first facial features of the patient, second facial features of a therapist, or a response to the material presented on the display;
at least one audio capture device configured to capture audio data for the ABT session related to at least one of a first voice of the patient or a second voice of the therapist; and
at least one processor configured to analyze, for the ABT session, data regarding the material presented on the display, the captured video data, and the captured audio data to produce an analysis of the ABT session.

2. The apparatus of claim 1, the display comprising a touch-sensitive display.

3. The apparatus of claim 1, further comprising at least one interactive accessory for which, when the interactive accessory is placed on a surface of the display, the apparatus identifies a position relative to the presented material.

4. The apparatus of claim 1, wherein the display is one of a tabletop display, a monitor, a projected display, or a wall mounted display.

5. The apparatus of claim 1, wherein the at least one processor configured to analyze the captured audio data is configured to:
identify at least one of the first voice associated with the patient or the second voice associated with the therapist; and
perform, for each identified voice, a tonal recognition on the captured audio data to determine if a tone of voice for the identified voice is one of a first tone of voice associated with a baseline stress level or a second tone of voice associated with a stress level above the baseline stress level.

6. The apparatus of claim 1, further comprising a presented-material capture device that captures the material presented for the ABT session to the patient.

7. The apparatus of claim 1, wherein the at least one processor configured to analyze the captured video data is configured to:
identify one of (1) a set of responses to the presented material, (2) a set of measures of patient agitation, or (3) a set of facial features.

8. The apparatus of claim 1, wherein the data regarding the material presented on the display, the captured video data, and the captured audio data are correlated by time.

9. The apparatus of claim 8, wherein the analysis of the ABT session comprises at least one of a first analysis of therapist efficacy, a second analysis of the efficacy of different material presented on the display, or a third analysis of a progress of the patient.

10. The apparatus of claim 9, wherein the at least one processor is further configured to produce at least one report regarding the analysis of the ABT session.

11. The apparatus of claim 1, wherein to analyze, for the ABT session, the data regarding the material presented on the display, the captured video data, and the captured audio data to produce the analysis of the ABT session, the at least one processor is configured to process at least one of the data regarding the material presented on the display, the captured video data, and the captured audio data using one of a machine-trained network or a neural network.

12. A method of providing automated analysis and monitoring of an applied behavioral therapy (ABT) session, the method comprising:
presenting material for the ABT session to a patient via a display;
capturing video data for the ABT session related to at least one of first facial features of the patient, second facial features of a therapist, or a response to the material presented;
capturing audio data for the ABT session related to at least one of a first voice of the patient or a second voice of the therapist; and
analyzing, for the ABT session, data regarding the material presented, the captured video data, and the captured audio data to produce an analysis of the ABT session.

13. The method of claim 12, wherein the display comprises a touch-sensitive display.

14. The method of claim 12, further comprising:
identifying, when at least one interactive accessory is placed on a surface of the display, a position of the at least one interactive accessory relative to the presented material.

15. The method of claim 12, wherein the display is one of a tabletop display, a monitor, a projected display, or a wall mounted display.

16. The method of claim 12, wherein analyzing the captured audio data comprises:
identifying at least one of the first voice associated with the patient or the second voice associated with the therapist; and
performing, for each identified voice, a tonal recognition on the captured audio data to determine if a tone of voice for the identified voice is one of a first tone of voice associated with a baseline stress level or a second tone of voice associated with a stress level above the baseline stress level.

17. The method of claim 12, further comprising capturing the material presented for the ABT session to the patient.

18. The method of claim 12, wherein analyzing the captured video data comprises:
identifying one of (1) a set of responses to the presented material, (2) a set of measures of patient agitation, or (3) a set of facial features.

19. The method of claim 12, wherein the data regarding the material presented on the display, the captured video data, and the captured audio data are correlated by time.

20. The method of claim 19, wherein the analysis of the ABT session comprises at least one of a first analysis of therapist efficacy, a second analysis of the efficacy of different material presented on the display, or third analysis of a progress of the patient based on more than one of the data regarding the material presented on the display, the captured video data, and the captured audio data.

21. The method of claim 20, further comprising:
producing at least one report regarding the analysis of the ABT session.

22. The method of claim 12, wherein analyzing, for the ABT session, data regarding the material presented on the display, the captured video data, and the captured audio data to produce the analysis of the ABT session comprises processing at least one of the data regarding the material presented on the display, the captured video data, and the captured audio data using one of a machine-trained network or a neural network.
